(19) United States
(12) Patent Application Publication
(10) Pub. No.: US 2023/0364294 A1
(43) Pub. Date: Nov. 16, 2023

(54) FRAGRANCE DEVICE FOR AUTOMOTIVE AIR DUCT

(71) Applicant: Ningbo Joysonquin Automotive Systems Holding Co., Ltd., Ningbo (CN)

(72) Inventors: Qiuhe REN, Ningbo (CN); Zan WANG, Ningbo (CN); Haodong HU, Ningbo (CN)

(21) Appl. No.: 17/876,627

(22) Filed: Jul. 29, 2022

(30) Foreign Application Priority Data
May 11, 2022 (CN) 202221119318.8

(51) Int. Cl. A61L 9/12; B60H 3/00

(52) U.S. Cl. CPC A61L 9/12; B60H 3/0028; A61L 2209/133; A61L 2209/15; A61L 2209/134

(57) ABSTRACT

A fragrance device for an automotive air duct includes: a plurality of air deflection openings, a plurality of fragrance units, and a plurality of air deflectors. The air deflection openings are disposed on one side of the automotive air duct. Each of the fragrance units corresponds to one of the air deflection openings. The fragrance unit includes a fragrance shell and a fragrance piece. The fragrance piece is disposed in the fragrance shell, and each side of the fragrance shell communicates with the air deflection opening. The air deflector is located at the air deflection opening, and the air deflector is rotatably connected between the fragrance shell and the automotive air duct. The different fragrance units have the different fragrance pieces. The air deflection opening is matched with the air deflector, and the air deflector in a horizontal state is capable of sealing the air deflection opening.

### CROSS-REFERENCE TO RELAYED APPLICATIONS

Pursuant to 35 U.S.C. § 119 and the Paris Convention Treaty, this application claims foreign priority to Chinese Patent Application No. 202221119318.8 filed May 11, 2022, the contents of which, including any intervening amendments thereto, are incorporated herein by reference. Inquiries from the public to applicants or assignees concerning this document or the related applications should be directed to: Matthias Scholl PC., Attn.: Dr. Matthias Scholl Esq., 245 First Street, 18th Floor, Cambridge, MA 02142.

### BACKGROUND

The disclosure relates to the field of automotive parts, and more particularly to a fragrance device for an automotive air duct.

With the rapid development of the automotive industry, the driving environment in a car has increasingly become a topic of public concern, and also has become an important concern for consumers to buy a car. A comfortable driving environment can reduce driver fatigue, stabilize emotions of the driver and contribute to driving pleasure. The existing fragrance device in a car can provide only one fragrance. If the user wants to switch to another fragrance, he has to manually replace the fragrance piece or replace the entire fragrance device. As a result, it is not convenient for the existing fragrance device to provide multiple fragrances.

### SUMMARY

To solve the aforesaid problems, the disclosure provides a fragrance device for an automotive air duct to provide more than one fragrance and easily switch one fragrance to another.

The fragrance device comprises a plurality of air deflection openings, a plurality of fragrance units and a plurality of air deflectors. The air deflection openings are disposed on one side of the automotive air duct. Each of the fragrance units corresponds to one of the air deflection openings. The fragrance unit comprises a fragrance shell and a fragrance piece. The fragrance piece is disposed in the fragrance shell, and each side of the fragrance shell communicates with the air deflection opening. The air deflector is disposed at the air deflection opening, and the air deflector is rotatably connected between the fragrance shell and the automotive air duct. The different fragrance units have the different fragrance pieces. The air deflection opening is matched with the air deflector, and the air deflector in a horizontal state is capable of sealing the air deflection opening.

This technical solution has the following technical effects: air flows in the automotive air duct. When the air deflector rotates, the fragrance of the fragrance piece in the fragrance shell is volatilized to the air deflection opening, so that the fragrance enters the automotive air duct. When the user wants to switch to another fragrance, the air deflector corresponding to the target fragrance is rotated, the other air deflectors are switched to the state of being flush with the air deflection openings, so that the volatilization of the other fragrances is prevented, which realizes rapid switching among fragrances and isolation among different fragrances. The user may switch among the fragrances according to the driving environment, which makes the driving environment more comfortable.

In a class of this embodiment, one side of the fragrance shell is provided with a fragrance piece mounting hole, the fragrance shell comprises a fragrance piece mounting slot therein, and the fragrance piece mounting hole communicates with the fragrance piece mounting slot.

This technical solution has the following technical effects: the fragrance piece is mounted into the fragrance piece mounting hole conveniently, and in addition, the position of the fragrance piece is limited in the fragrance piece mounting slot, which prevents the fragrance piece from falling down and facilitates volatilization of the fragrance.

In a class of this embodiment, the fragrance device for an automotive air duct further comprises a casing and at least one baffle. The casing and the baffle form a plurality of mounting spaces, and the fragrance unit is disposed in the mounting space.

This technical solution has the following technical effects: the casing and the baffle may locate the fragrance unit, so that the fragrance shell can be aligned with the air deflector and the air deflection opening when the fragrance shell is mounted to the automotive air duct. In addition, the fragrance unit can be replaced independently, so that there is no need to demount the whole fragrance device for an automotive air duct, which improves the efficiency of replacing the fragrance unit.

In a class of this embodiment, the fragrance device for an automotive air duct further comprises a base comprising a clamping groove. The clamping groove surrounds the plurality of fragrance units and forms a fit with the casing, and/or the clamping groove is disposed between the adjacent fragrance units and forms a fit with the baffle.

This technical solution has the following technical effects: the base is fixed through the clamping groove and supports the fragrance units and the casing, which improves the stability of the fragrance unit and the casing. In this way, when shakes occur due to the rotation of the air deflector or a running vehicle, the automotive air duct can still be connected with the casing stably.

In a class of this embodiment, the fragrance device for an automotive air duct further comprises a first reinforcer. The first reinforcer is disposed on an outer side of the casing. The first reinforcer is connected to the base.

This technical solution has the following technical effects: the first reinforcer further supports the casing from the side surface of the casing, which increases the connection strength between the casing and the base and improves the stability of the fragrance device for an automotive air duct.

In a class of this embodiment, the fragrance device for an automotive air duct further comprises a first limiting member and a second limiting member. The first limiting member and the second limiting member are clamped on two opposite sides of the casing. The first limiting member and the second limiting member are connected to the automotive air duct.

This technical solution has the following technical effects: the first limiting member and the second limiting member can laterally lock the casing and the automotive air duct, which prevents the automotive air duct from being separated from the casing along the horizontal direction. Meanwhile, the first limiting member is connected to the casing, and the second limiting member is also connected to the casing, so that the casing and the automotive air duct can form a close fit to clamp the rotating shaft of the air deflector, which improves the stability of rotation of the air deflector.

In a class of this embodiment, the fragrance device for an automotive air duct further comprises a drive assembly comprising a drive assembly body and an output shaft. The drive assembly body is connected to one side of the fragrance shell, and the output shaft is disposed through the fragrance shell and is connected to the air deflector.

This technical solution has the following technical effects: the output shaft of the drive assembly is connected to the air deflector to drive the air deflector to rotate, so that the fragrance is volatilized into the automotive air duct. The plurality of air deflectors is controlled by the plurality of drive assemblies, so that intelligent switching of actions of the air deflectors can be realized, which facilitates switching among fragrances.

In a class of this embodiment, the fragrance device for an automotive air duct further comprises a control assembly comprising a control assembly shell. The control assembly shell is disposed on one side of the fragrance shell, and the drive assembly is connected to the control assembly shell.

This technical solution has the following technical effects: the control assembly is used to control the actions of the drive assemblies, that is, to control the actions of the air deflectors, thereby realizing volatilization and switching of the fragrance.

In a class of this embodiment, the fragrance unit further comprises a fragrance volatilizing member. The fragrance volatilizing member is disposed in the fragrance shell and disposed on one side of the fragrance piece. The fragrance volatilizing member is electrically connected to the control assembly.

This technical solution has the following technical effects: the fragrance volatilizing member is used to promote volatilization of the fragrance of the fragrance piece, for example, to further improve the fragrance effect in the automotive air duct by heating. The fragrance volatilizing member is controlled by the control assembly, so that the fragrance volatilizing member can be turned on or off intelligently and the degree of volatilization of the fragrance can be adjusted.

In a class of this embodiment, the fragrance device for an automotive air duct further comprises a sterilization assembly. The sterilization assembly is disposed in the automotive air duct and disposed on the other side of the automotive air duct opposite to the air deflection opening.

This technical solution has the following technical effects: the sterilization assembly is used to sterilize the air flowing in the automotive air duct. When the sterilization assembly is faced with the air deflection opening, bacteria in the air coming from the fragrance unit can be effectively removed by the sterilization assembly, which makes the air flowing in the automotive air duct healthier.

Based on the above, the above technical solutions of the present application may have one or more of the following advantages or beneficial effects: i) Air flows in the automotive air duct, and the drive assembly controls the air deflector to rotate, thereby promoting the air exchange between the fragrance unit and the automotive air duct. As a result, the target fragrance can enter the automotive air duct through the corresponding air deflection opening, and the other air deflectors are switched to the state of being flush with the air deflection opening, so that the volatilization of the other fragrances is prevented, which realizes rapid switching among fragrances and isolation among different fragrances, thereby facilitating switching among the fragrances. ii) The fragrance volatilizing member is used to promote volatilization of the fragrance of the fragrance piece, for example, to further improve the fragrance effect in the automotive air duct by heating. The fragrance volatilizing member is controlled by the control assembly, so that the fragrance volatilizing member can be turned on or off intelligently and the degree of volatilization of the fragrance can be adjusted. iii) The sterilization assembly is used to sterilize the air flowing in the automotive air duct. When the sterilization assembly is faced with the air deflection opening, bacteria in the air coming from the fragrance unit can be effectively removed, which makes the air flowing in the automotive air duct healthier.

### BRIEF DESCRIPTION OF THE DRAWINGS

In order to more clearly illustrate the technical solutions of the embodiments of the disclosure, the accompanying drawings used in the description of the embodiments will be briefly described below. It is apparent that the accompanying drawings in the following description are only some embodiments of the disclosure. Those of ordinary skill in the art can obtain other accompanying drawings according to these drawings without any creative work.

FIG. 1 is a schematic structural diagram of a fragrance device for an automotive air duct according to an embodiment of the disclosure;

FIG. 2 is an exploded view of the fragrance device for the automotive air duct in FIG. 1;

FIG. 3 is a schematic diagram showing connection between fragrance units and a base in FIG. 2; and

FIG. 4 is a schematic structural diagram of the automotive air duct in FIG. 2.

In the drawings, the following reference numbers are used: 100. Fragrance device for automotive air duct; 110. Automotive air duct; 111. First limiting member; 112. Second limiting member; 120. Air deflection opening; 130. Fragrance unit; 131. Fragrance shell; 132. Fragrance piece; 133. Fragrance piece mounting hole; 134. Fragrance volatilizing member; 135. Limiting frame; 136. Air chamber; 140. Air deflector; 150. Casing; 151. Baffle; 152. Mounting space; 153. Insertion member; 154. First reinforcer; 160. Base; 161. Clamping groove; 161a. Insertion port; 170. Drive assembly; 180. Control assembly; 181. Second reinforcer; and 190. Sterilization assembly.

### DETAILED DESCRIPTION OF THE EMBODIMENTS

To further illustrate the disclosure, embodiments detailing a fragrance device of the disclosure are described below. It should be noted that the following embodiments are intended to describe and not to limit the disclosure.

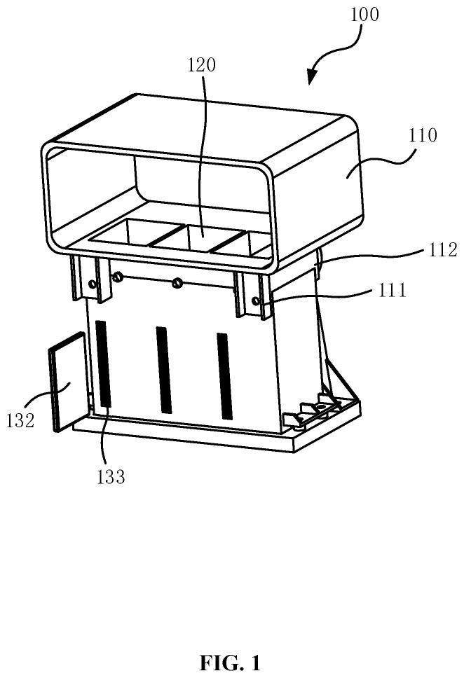

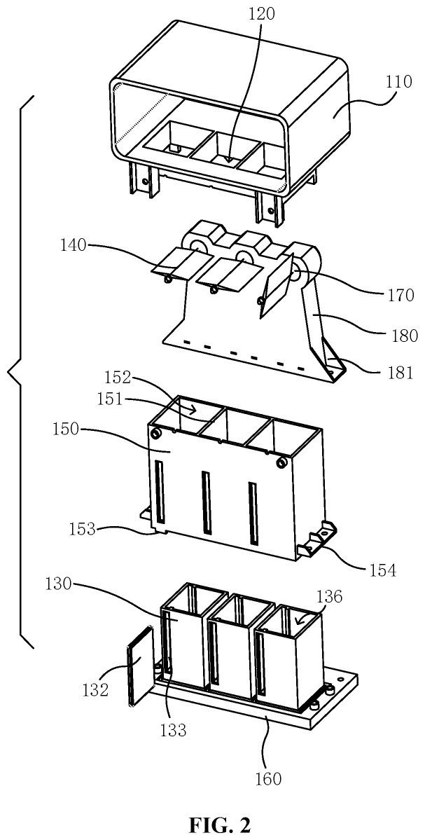

Referring to FIGS. 1-4, an embodiment of the disclosure provides a fragrance device 100 for an automotive air duct 110. The fragrance device comprises a plurality of air deflection openings 120, a plurality of fragrance units 130 and a plurality of air deflectors 140. The air deflection openings 120 are disposed on one side of the automotive air duct 110. Each of the fragrance units 130 corresponds to one of the air deflection openings 120. The fragrance unit 130 comprises a fragrance shell 131 and a fragrance piece 132. The fragrance piece 132 is disposed in the fragrance shell 131, and each side of the fragrance shell 131 communicates with the air deflection opening 120. The air deflector 140 is disposed at the air deflection opening 120, and the air deflector 140 is rotatably connected between the fragrance shell 131 and the automotive air duct 110. The different fragrance units 130 have the different fragrance pieces 132. The air deflection opening 120 is matched with the air deflector 140, and the air deflector 140 in a horizontal state is capable of sealing the air deflection opening 120.

In this embodiment, air flows in the automotive air duct 110, and the automotive air duct 110 may communicate with any vent duct in the driving environment. When the air deflector 140 rotates, the fragrance of the fragrance piece 132 in the fragrance shell 131 may be volatilized to the air deflection opening 120, so that the fragrance enters the automotive air duct 110 and then enters the driving environment. When the user wants to switch to another fragrance, the air deflector 140 corresponding to the target fragrance is rotated, the other air deflectors 140 are switched to the state of being flush with the air deflection openings 120, so that the volatilization of the other fragrances is prevented, which realizes rapid switching among fragrances and isolation among different fragrances. The user may switch among the fragrances according to the driving environment, which is more humanized and makes the driving environment more comfortable.

Preferably, the plurality of air deflection openings 120 is disposed at the bottom of the automotive air duct 110, and the plurality of fragrance units 130 communicate with the bottom of the automotive air duct 110 and support the automotive air duct 110.

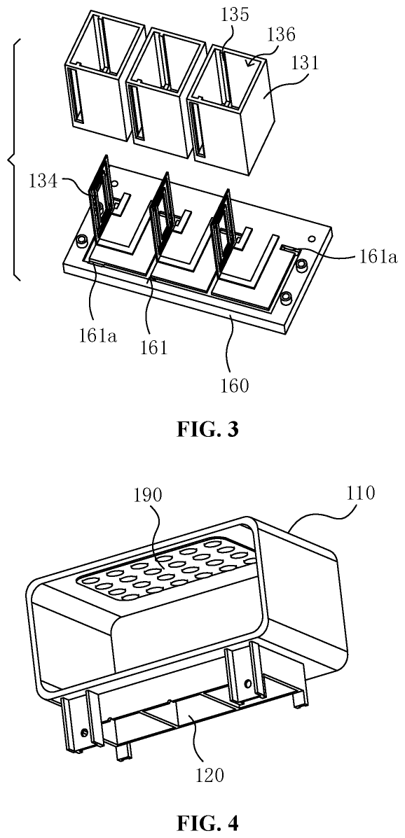

In a specific embodiment, referring to FIG. 3, one side of the fragrance shell 131 is provided with a fragrance piece mounting hole 133, the fragrance shell 131 comprises a fragrance piece mounting slot therein, and the fragrance piece mounting hole 133 communicates with the fragrance piece mounting slot.

Preferably, the fragrance piece 132 has a plate-like structure, so that the fragrance piece 132 with a fixed mass may have a larger surface area, which makes the fragrance volatilized more efficiently.

Further, the fragrance piece mounting hole 133 is, for example, a vertical slotted hole, and the fragrance piece 132 is vertically mounted into the fragrance shell 131. At this time, the air coming from the air deflection opening 120 may flow in a direction parallel to the fragrance piece 132, so that the fragrance can be volatilized without hindrance.

Furthermore, a limiting frame 135, for example, is disposed in the fragrance shell 131. The limiting frame 135 is disposed on one side of the fragrance piece mounting hole 133, and the fragrance piece mounting slot is formed between the limiting frame 135 and a side plate of the fragrance shell 131, so that the position of the fragrance piece 132 is limited and the fragrance piece 132 is more stable. An air chamber 136 is formed on the side of the limiting frame 135 away from the fragrance piece mounting slot, and the limiting frame 135 has a fragrance emission opening therein. The fragrance piece 132 emits the fragrance to the air chamber 136 through the limiting frame 135, so that the fragrance enters the air deflection opening 120 along with the air flow.

In a specific example, the fragrance device for an automotive air duct 100 further comprises, for example, a casing 150 and at least one baffle 151. The casing 150 and the baffle 151 form a plurality of mounting spaces 152, and the fragrance unit 130 is disposed in the mounting space 152. The casing 150 and the baffle 151 may locate the fragrance unit 130, so that the fragrance shell 131 can be aligned with the air deflector 140 and the air deflection opening 120 when the fragrance shell 131 is mounted to the automotive air duct 110. In addition, after the casing 150 is fixed, the fragrance shell 131 will not be offset. When the user wants another fragrance for the fragrance unit 130 or when the fragrance unit 130 needs to be repaired, the fragrance unit 130 may be replaced independently instead of demounting the whole fragrance device for an automotive air duct 100, which improves the efficiency of replacing the fragrance unit 130.

In a specific example, the fragrance device for an automotive air duct 100 further comprises, for example, a base 160. The base 160 comprises a clamping groove 161. The clamping groove 161 surrounds the plurality of fragrance units 130 and forms a fit with the casing 150, and/or the clamping groove 161 is disposed between the adjacent fragrance units 130 and forms a fit with the baffle 151. The base 160 is fixed in the horizontal direction through the clamping groove 161, and moreover, the base 160 is capable of stably supporting the fragrance unit 130 and the casing 150, which improves the stability of the fragrance unit 130 and the casing 150. In this way, when shakes occur due to the rotation of the air deflector 140 or a running vehicle, the automotive air duct 110 can still be connected with the casing 150 stably.

Preferably, clamping groove 161 is, for example, further provided with an insertion port 161a. The insertion port 161a is disposed at least on two opposite ends of the clamping groove 161. Correspondingly, the casing 150 is, for example, provided with an insertion member 153 matched with the insertion port 161a. The insertion member 153 is disposed on two opposite ends at the bottom of the casing 150. The insertion port 161a is matched with the insertion member 153, which increases the connection strength between the casing 150 and the base 160.

In a specific embodiment, the fragrance device for an automotive air duct 100 further comprises, for example, a first reinforcer 154. The first reinforcer 154 is disposed on an outer side of the casing 150, and the first reinforcer 154 is connected to the base 160. For example, the first reinforcer 154 comprises a reinforcing rib along the longitudinal direction and a fastener mounting plate along the transverse direction. The fastener mounting plate is further connected to the base 160 through fasteners such as screws, which further increases the connection strength between the casing 150 and the base 160 and improves the stability of the fragrance device for an automotive air duct 100.

In a specific embodiment, the fragrance device for an automotive air duct 100 further comprises a first limiting member 111 and a second limiting member 112. The first limiting member 111 and the second limiting member 112 are clamped on two opposite sides of the casing 150. The first limiting member 111 and the second limiting member 112 are connected to the automotive air duct 110. The first limiting member 111 and the second limiting member 112 can laterally lock the casing 150 and the automotive air duct 110, which prevents the automotive air duct 110 from being separated from the casing 150 along the horizontal direction. Meanwhile, the first limiting member 111 is connected to the casing 150, and the second limiting member 112 is also connected to the casing 150, so that the casing 150 and the automotive air duct 110 can form a close fit to clamp the rotating shaft of the air deflector 140, which improves the stability of rotation of the air deflector 140.

Preferably, the first limiting member 111 is provided with a mounting hole, the casing 150 is provided with a corresponding threaded rod, and the first limiting member 111 may be connected to the casing 150 through a screw, which further increases the connection strength between the automotive air duct 110 and the casing 150.

Preferably, a length of the first limiting member 111 extending toward the base 160 is greater than a length of the second limiting member 112 extending toward the base 160. In this case, when the automotive air duct 110 is to be assembled with the casing 150, the automotive air duct 110 may be leaned toward the first limiting member 111 such that the mounting hole forms a fit with the threaded rod, and then the second limiting member 112 is clamped with the casing 150, so that the automotive air duct 110 can be assembled with the casing 150 conveniently.

In a specific embodiment, the fragrance device for an automotive air duct 100 further comprises, for example, a drive assembly 170. The drive assembly 170 comprises a drive assembly 170 body and an output shaft. The drive assembly 170 body is connected to one side of the fragrance shell 131, and the output shaft is disposed through the fragrance shell 131 and is connected to the air deflector 140. For example, the drive assembly 170 is, for example, a motor. The rotating shaft of the air deflector 140 is rotatably connected to a top of the casing 150, and the output shaft is connected to the rotating shaft of the air deflector 140, so that the air deflector 140 is driven to rotate, which makes the fragrance volatilized into the automotive air duct 110.

The plurality of air deflectors 140 is respectively controlled by the plurality of drive assemblies 170, so that the air deflectors 140 may have different actions, thereby realizing intelligent switching among fragrances.

In a specific embodiment, the fragrance device for an automotive air duct 100 further comprises, for example, a control assembly 180. The control assembly 180 comprises a control assembly shell. The control assembly shell is disposed on one side of the fragrance shell 131, and the drive assembly 170 is connected to the control assembly shell. For example, the drive assembly 170 is mounted in the control assembly shell, and the control assembly 180 controls the action of the drive assembly 170 so as to control the action of the air deflector 140, thereby realizing volatilization of the fragrances and switching among the fragrances.

In a specific embodiment, the fragrance device for an automotive air duct 100 further comprises, for example, a second reinforcer 181. The second reinforcer 181 is disposed on two opposite sides of the control assembly shell. The second reinforcer 181 also comprises, for example, a reinforcing rib along the longitudinal direction and a fastener mounting plate along the transverse direction, thereby further supporting the control assembly 180.

In a specific embodiment, the fragrance unit 130 further comprises, for example, a fragrance volatilizing member 134. The fragrance volatilizing member 134 is disposed in the fragrance shell 131 and disposed on one side of the fragrance piece 132, and the fragrance volatilizing member 134 is electrically connected to the control assembly 180. For example, the fragrance volatilizing member 134 is an electric heating member which promotes volatilization of the fragrance of the fragrance piece 132 by heating, thereby improving the fragrance effect in the automotive air duct 110. The electric heating member may be disposed on a side of the fragrance piece 132 away from the limiting frame 135. A power cord of the electric heating member may be connected to the control assembly 180 along the bottom of the fragrance shell 131 to give way to the air in the air chamber 136, which avoids affecting the volatilization of the fragrance.

When the user selects a fragrance, the control assembly 180 controls the corresponding fragrance volatilizing member 134 to be energized, the corresponding air deflector 140 to rotate and the other fragrance volatilizing members 134 and the other air deflectors 140 not to work, so that the fragrance units 130 can be turned on or off intelligently. Optionally, the control assembly 180 may also adjust the power of the fragrance volatilizing member 134 or the rotational speed of the air deflector 140, thereby adjusting the degree of volatilization of the fragrance.

In a specific embodiment, referring to FIG. 4, the fragrance device for an automotive air duct 100 further comprises, for example, a sterilization assembly 190. The sterilization assembly 190 is disposed in the automotive air duct 110 and disposed on the other side of the automotive air duct 110 opposite to the air deflection opening 120. For example, the sterilization assembly 190 may be an ultraviolet sterilization lamp used to sterilize the air flowing in the automotive air duct 110. When the sterilization assembly 190 is faced with the air deflection opening 120, bacteria in the air coming from the fragrance unit 130 can be effectively removed by the sterilization assembly 190, which makes the air flowing in the automotive air duct 110 healthier.

The inner wall of the automotive air duct 110 may act as a lamp shade to prevent ultraviolet light from being transmitted outward through the automotive air duct 110 and affecting the human body.

It will be obvious to those skilled in the art that changes and modifications may be made, and therefore, the aim in the appended claims is to cover all such changes and modifications.

What is claimed is:

1. A fragrance device, being connected to an automotive air duct when in use, the fragrance device comprising:
a plurality of air deflection openings, disposed on one side of the automotive air duct;
a plurality of fragrance units, each corresponding to one of the air deflection openings, wherein each of the plurality of fragrance units comprises a fragrance shell and a fragrance piece, the fragrance piece is disposed in the fragrance shell, and each side of the fragrance shell communicates with the air deflection openings; and
a plurality of air deflectors, wherein the plurality of air deflectors is disposed at the plurality of air deflection openings, respectively, and the plurality of air deflector is rotatably connected between the fragrance shell and the automotive air duct; wherein
the plurality of fragrance units is provided with different fragrance pieces, respectively; and the plurality of air deflection openings is matched with the plurality of air deflectors, respectively, and the plurality of air deflectors in a horizontal state is capable of sealing the air deflection openings, respectively.

2. The fragrance device of claim 1, wherein one side of the fragrance shell is provided with a fragrance piece mounting hole, the fragrance shell comprises a fragrance piece mounting slot therein, and the fragrance piece mounting hole communicates with the fragrance piece mounting slot.

3. The fragrance device of claim 1, wherein the fragrance device further comprises a casing and at least one baffle, the casing and the baffle form a plurality of mounting spaces, and the plurality of fragrance units is disposed in the mounting spaces, respectively.

4. The fragrance device of claim 3, wherein the fragrance device further comprises a base comprising a clamping groove;
the clamping groove surrounds the plurality of fragrance units and forms a fit with the casing, and/or the clamping groove is disposed between the adjacent fragrance units and forms a fit with the baffle.

5. The fragrance device of claim 4, wherein the fragrance device further comprises a first reinforcer; the first reinforcer is disposed on an outer side of the casing, and the first reinforcer is connected to the base.

6. The fragrance device of claim 3, wherein the fragrance device further comprises a first limiting member and a second limiting member; the first limiting member and the second limiting member are clamped on two opposite sides of the casing, and the first limiting member and the second limiting member are connected to the automotive air duct.

7. The fragrance device of claim 1, wherein the fragrance device further comprises a drive assembly comprising a drive assembly body and an output shaft;
the drive assembly body is connected to one side of the fragrance shell, and the output shaft is disposed through the fragrance shell and is connected to the air deflector.

8. The fragrance device of claim 7, wherein the fragrance device further comprises a control assembly comprising a control assembly shell; the control assembly shell is disposed on one side of the fragrance shell, and the drive assembly is connected to the control assembly shell.

9. The fragrance device of claim 8, wherein the plurality of fragrance units further comprises:
a fragrance volatilizing member; the fragrance volatilizing member is disposed in the fragrance shell and disposed on one side of the fragrance piece, and the fragrance volatilizing member is electrically connected to the control assembly.

10. The fragrance device of claim 1, wherein the fragrance device further comprises:
a sterilization assembly; the sterilization assembly is disposed in the automotive air duct and disposed on the other side of the automotive air duct opposite to the plurality of air deflection openings.
